(19) United States
(12) Patent Application Publication
(10) Pub. No.: US 2013/0071450 A1
(43) Pub. Date: Mar. 21, 2013

(54) GELS FOR TRANSDERMAL DELIVERY

(71) Applicant: Covidien LP, Mansfield, MA (US)

(72) Inventor: Warren Copp-Howland, Chicopee, MA (US)

(73) Assignee: COVIDIEN LP, Mansfield, MA (US)

(21) Appl. No.: 13/677,831

(22) Filed: Nov. 15, 2012

Related U.S. Application Data
(62) Division of application No. 12/872,539, filed on Aug. 31, 2010, now abandoned.
(60) Provisional application No. 61/315,145, filed on Mar. 18, 2010.

(51) Int. Cl. A61K 47/32

(52) U.S. Cl. CPC A61K 47/32
USPC 424/400; 514/343; 514/163; 514/629; 514/282; 514/536; 514/330; 514/758; 514/759; 514/626; 514/239.2; 514/54; 424/744; 424/752; 424/729; 424/728; 424/766; 424/725

(57) ABSTRACT

The present disclosure provides hydrogels that are suitable for drug delivery. In embodiments, hydrogels of the present disclosure may be used for transdermal delivery of bioactive agents, including drugs. The hydrogels of the present disclosure may also be useful as conductive compositions for use with electrodes.

### CROSS REFERENCE TO RELATED APPLICATIONS

This application is a divisional application of U.S. patent application Ser. No. 12/872,539, filed on Aug. 31, 2010 which, in turn, claims the benefit of, and priority to, U.S. Provisional Patent Application Ser. No. 61/315,145 filed on Mar. 18, 2010, the entire disclosures of each of which are incorporated by reference herein for all purposes.

### TECHNICAL FIELD

The present disclosure relates to gels suitable for drug delivery. In embodiments, a gel of the present disclosure may be a hydrogel which includes components which enhance the delivery of drugs through the skin.

### BACKGROUND

Hydrogels constitute a broad class of materials which may be completely water soluble or swell extensively in water but are not completely water soluble. They have been used in a variety of biomedical applications and may be applied in bulk forms which vary from clear to opaque, and from a relatively stiff to a relatively soft consistency. Sometimes the bulk forms include reinforcement members which may be woven or non-woven fabrics to increase the composite strength and/or dimensional stability of the bulk form.

Hydrogels have been used as coatings for various biomedical applications. They have also been used as adhesives and/or sealants, and for the delivery of bioactive agents, including drugs.

Drugs may be delivered to a patient by many routes, including orally, buccally, intravenously, intramuscularly, parenterally, subcutaneously, sublingually topically, combinations thereof, and the like. Different routes of administration may present different challenges for effective administration of bioactive agents. For example, the stratum corneum layer of the skin may limit the effectiveness of transdermal delivery of bioactive agents.

Improved hydrogels for drug delivery, including those which may be used for the transdermal delivery of bioactive agents, remain desirable.

### SUMMARY

The present disclosure provides hydrogels that are suitable for the delivery of bioactive agents, including drugs. Methods for delivering bioactive agents using the hydrogels of the present disclosure are also provided. In embodiments, the hydrogels of the present disclosure may also be conductive, and thus may also be suitable for use as a conductive composition with an electrode, including a medical electrode.

In embodiments, a hydrogel composition of the present disclosure includes a polymeric component such as gelatin, polysaccharides, crosslinked acrylamide polymers, hydroxyethylmethacrylate polymers, crosslinked polyhydroxyethylacrylate, polymerized, crosslinked 2-acrylamido-2-methylpropane sulfonic acid polymers, crosslinked polyvinylpyrrolidone, polyacrylic acid, copolymers of the foregoing, one or more salts thereof, and combinations thereof; at least one penetration enhancer such as sulfoxides, alcohols, pyrrolidones, laurocapram, solvents, fatty alcohols, amides, amino acids, azones, oils, fatty acids and their esters, macrocycles, phospholipids, glycols, and combinations thereof; and at least one bioactive agent.

Methods of the present disclosure include, in embodiments, contacting a tissue of an animal with a hydrogel of the present disclosure; allowing the hydrogel to adhere to the tissue; and releasing a bioactive agent from the hydrogel.

Medical electrodes are also provided. In embodiments, a medical electrode of the present disclosure includes a substrate; a conductive composition on at least a portion of a surface of the substrate, the conductive composition including at least one hydrogel including: a polymeric component such as gelatin, polysaccharides, crosslinked acrylamide polymers, hydroxyethylmethacrylate polymers, crosslinked polyhydroxyethylacrylate, polymerized, crosslinked 2-acrylamido-2-methylpropane sulfonic acid polymers, crosslinked polyvinylpyrrolidone, polyacrylic acid, copolymers of the foregoing, one or more salts thereof, and combinations thereof; at least one penetration enhancer such as sulfoxides, alcohols, pyrrolidones, laurocapram, solvents, fatty alcohols, amides, amino acids, azones, oils, fatty acids and their esters, macrocycles, phospholipids, glycols, and combinations thereof; and at least one bioactive agent.

Other methods of the present disclosure include, in embodiments, contacting a tissue of an animal with a medical electrode possessing a hydrogel of the present disclosure; allowing the hydrogel to adhere to the tissue; and releasing a bioactive agent from the hydrogel.

### DETAILED DESCRIPTION

Any adhesive application, including those involving tissue, are within the purview of the hydrogel compositions of the present disclosure. In embodiments, hydrogels may be utilized as adhesives and/or devices for the delivery of bioactive agents. In some embodiments, the hydrogels may also be used as conductive compositions with medical electrodes.

As used herein, the term “hydrogel” may refer to a wide variety of polymer-based compositions. These materials may be synthesized, for example, from monomer(s), or from monomer(s) mixed with polymer(s) or cross-linked polymer solutions in water. They may be obtained by chemical modification of existing polymer(s), or by adding water to existing dry polymers.

Any biocompatible hydrogel may be utilized in accordance with the present disclosure. Generally speaking, a hydrogel according to the present disclosure may include a coherent, three-dimensional aqueous polymer system capable of imbibing water without liquefying. In embodiments, insolubility in water may be provided by crosslinking a hydrogel polymer. In embodiments, hydrogels or water-containing gels of the present disclosure may include water and various polymeric components including gelatin; polysaccharides; crosslinked acrylamide polymers; hydroxyethylmethacrylate polymers; crosslinked polyhydroxyethylacrylate; polymerized, crosslinked 2-acrylamido-2-methylpropane sulfonic acid polymers, or one of their salts such as the sodium or potassium type; crosslinked polyvinylpyrrolidone; polyacrylic acid; copolymers of the aforementioned monomers with each other; copolymers of the aforementioned monomers with other polymers such as polystyrene or other non-hydrogel-forming polymers; one or more salts of the foregoing; and combinations thereof.

For example, by cross-linking homopolymers of an acrylamide derivative such as 2-acrylamido-2-methylpropanesulfonic acid or one of its salts, hydrogels may be formed. Copolymers thereof may also be formed in the same way with acrylamide. Cross-linked homopolymers of acrylic acid and of methacrylic acid, their salts and copolymers thereof, may similarly form hydrogels, as do other acrylic cross-linked homopolymers and copolymers.

Hydrogels of the present disclosure derive their adhesive properties in part from their ability to absorb water. When a relatively dry body of hydrogel contacts moisture, such as the moisture in tissue, particularly internal tissue, or any other moist surface, it develops an aggressive adhesive nature. When the polymer of the hydrogel is crosslinked to an adequate degree, the bulk hydrogel is strong enough, even when swelled with additional liquid, to adhere to tissue and thus remain affixed to skin for delivery of a bioactive agent. The hydrogel may also be strong enough at this point to provide adhesive support for pacing leads where utilized as a conductive composition with an electrode, thereby establishing extended connection of the lead to tissue.

In use, a hydrogel of the present disclosure may contain the polymer or copolymer, and any other additives, including components utilized to form the copolymer and/or crosslinking agent(s), polymerization initiator(s), electrolyte(s), bioactive agent(s), neutralizer(s), thickener(s), penetration enhancer(s), combinations thereof, and the like, in an amount from about 4% by weight to about 97% by weight of the hydrogel, in embodiments from about 20% by weight to about 60% by weight of the hydrogel, with the balance being water and/or a humectant, combinations thereof, and the like. In embodiments, a hydrogel may contain the polymer or copolymer, and any other additives in an amount of about 20%.

In embodiments, a first monomer which may be used to form a copolymer for use in a hydrogel includes acrylic acid, a salt thereof, or a mixture thereof. The copolymer thus produced by polymerization includes acid acrylate moieties (—CO₂H and/or —CO₂M, in which M is a cation such as sodium ion, potassium ion, lithium ion, ammonium or substituted ammonium ion, etc.) directly attached to the polymer backbone.

In embodiments, a copolymer utilized in a hydrogel of the present disclosure may include a second monomer which may be one of more monomers selected from CH₂═CHC(O)XR, in which X is O or NH, and R is an unsubstituted or substituted alkyl group of 1 to 5 carbon atoms. The polymer produced by this polymerization includes groups of the structure —C(O)XR directly attached to the polymer backbone.

Suitable unsubstituted alkyl groups include methyl, ethyl, n-propyl, n-butyl, and n-pentyl. Suitable substituents that may be present in a substituted alkyl group are halo (such as F, Cl, or Br) cyano, carboxylic acid and salts thereof (i.e., —CO₂H or —CO₂M, in which M is a cation), phosphate and salts thereof, and sulfonic acid and salts thereof. An example of such a substituted alkyl group is (3-sulfopropyl)acrylic acid ester, potassium salt. Suitable second monomers include 2-acrylamido-2-methylpropane sulfonic acid (CH₂═CH—CONHC(CH₃)₂—CH₂—SO₃H) and/or a salt thereof. Suitable salts include the sodium, lithium, potassium, ammonium, and substituted ammonium salts, and mixtures thereof.

In embodiments, the second monomer utilized in a copolymer component of a hydrogel of the present disclosure is 2-acrylamido-2-methylpropane sulfonic acid sodium salt (NaAMPS) (CH₂═CH—CONHC(CH₃)₂—CH₂—SO₃⁻M⁺). Thus, in some embodiments, the first monomer utilized in a copolymer component of a hydrogel of the present disclosure may include a mixture of acrylic acid and sodium acrylate, and the second monomer may include sodium 2-acrylamido-2-methylpropane sulfonate.

The first monomer (acrylic acid and/or salt or salt thereof, calculated as acrylic acid) may be present in an amount of from about 8 percent by weight (wt %) to about 85 wt % of the copolymer, in embodiments from about 10 wt % to about 80 wt % of the copolymer, of the total amount of the copolymer in the hydrogel. The second monomer, in embodiments NaAMPS, may be present in an amount of from about 15 wt % to about 92 wt % of the copolymer, in embodiments from about 20 wt % to about 90 wt % of the copolymer.

Excessive crosslinking decreases the tack of the hydrogel. Too little crosslinking decreases its cohesive strength. Thus, in embodiments, a crosslinking agent may be utilized in forming the polymer suitable as a hydrogel of the present disclosure. The cross-linking agent or mixture of cross-linking agents may be utilized in an effective amount. An effective amount of cross-linking agent is an amount that produces a hydrogel with the desired physical properties, such as coherence and adhesion to skin, and/or electrical properties. The amount required will depend on, for example, the molecular weight of the cross-linking agent, the number of ethylenically unsaturated, free radical polymerizable groups present in the cross-linking agent, and the amount of free radical polymerizable monomers present in the monomer mix. When the cross-linking agent is present, the amount of crosslinking agent will be present in an amount of from about 0.01 wt % to 1 wt % of the copolymer utilized in the hydrogel, in embodiments from about 0.02 wt % to 0.08 wt % of the copolymer utilized in the hydrogel.

In embodiments, a polymerization initiator may be utilized with the first monomer and second monomer to form a copolymer for use in a hydrogel of the present disclosure. An effective amount of a polymerization initiator may be combined with the monomers to form such a copolymer. As used herein, an effective amount is an amount that produces efficient polymerization of the monomers under polymerization conditions to produce a hydrogel suitable for use as a drug delivery device and/or conductive composition for use with a medical electrode. Numerous free radical polymerization initiators are within the purview of those skilled in the art. The polymerization initiator may be a single compound or a mixture of compounds. Thermal and/or photo free radical polymerization initiators, for example, may be used.

In embodiments, suitable cross-linking agents include free radical polymerizable monomers that possess more than one ethylenically unsaturated, free radical polymerizable group. Numerous crosslinking agents polymerizable by free-radical initiated polymerization are within the purview of those skilled in the art. Crosslinking agents include, for example, bis-acrylamides and methacrylamides, such as N,N′-methylene bis-acrylamide; acrylate and methacrylate esters of polyols, such as ethylene glycol diacrylate and dimethacrylate, diethylene glycol diacrylate and dimethacrylate, trimethylolpropane triacrylate and trimethacrylate, ethoxylated trimethylolpropane triacrylate and trimethacrylate, pentaerythritol triacrylate and trimethacrylate, pentaerythritol tetraacrylate and tetramethacrylate, and polyethylene glycol diacrylates and dimethacrylates, such as the diacrylates and dimethacrylates of polyethylene glycols having a molecular weight of from about 200 to about 600, in embodiments from about 300 to about 500. In embodiments, a suitable crosslinking agent may include N,N′-methylene bis-acrylamide [(CH₂═CHCONH)₂CH₂].

Suitable thermal free radical polymerization initiators include azo compounds, such as 2,2-azobisisobutyronitrile (AIBN). Suitable photo free radical polymerization initiators are disclosed in “Photoinitiators for Free-Radical-Initiated Photoimaging Systems,” by B. M. Monroe and G. C. Weed, Chem. Rev., 93, 435-448 (1993) and in “Free Radical Polymerization” by K. K. Dietliker, in Chemistry and Technology of UV and EB Formulation for Coatings, Inks, and Paints, P. K. T. Oldring, ed., SITA Technology Ltd., London, 1991, Vol. 3, pp. 59-525. Suitable free radical photo polymerization initiators include, for example, 1-hydroxycyclohexylphenyl ketone (HCPK, IRGACURE® 184); 2-hydroxy-2-methyl-1-phenylpropan-1-one (DAROCUR® 1173); 2-hydroxy-1-[4-(2-hydroxyethoxy)phenyl]-2-methyl-1-propan-1-one (IRGACURE®2959), 2,2-dimethoxy-2-phenylacetophenone (benzildimethyl ketal, BDK, IRGACURE®651), benzophenone, a mixture of 50 wt % benzophenone and 50 wt % of 1-hydroxycyclohexylphenyl ketone (IRGACURE® 500), and combinations thereof.

The polymerization initiator may be present in a copolymer utilized in a hydrogel in an amount less than about 1 wt % of the copolymer, in embodiments less than about 0.7 wt % of the copolymer, in other embodiments less than about 0.4 wt % of the copolymer.

In addition to a free radical initiator, free radical polymerization inhibitors may be present with one or more of the monomers, and/or the crosslinking agent, and/or may be added to the mixture to prevent premature polymerization of the reaction mixture. Suitable free radical polymerization inhibitors include, for example, hydroquinone, 4-methoxyphenol, di-t-butyl-p-cresol, pyrogallol, t-butyl catechol, benzoquinone, 4,4′-thio-bis-(3-methyl-6-t-butylphenol), and 2,2′-methylene-bis-(4-methyl-6-t-butylphenol). When present, the amount of the polymerization inhibitor may be from about 0.01 wt % to about 5 wt % of the hydrogel, in embodiments from about 1 wt % to about 4 wt % of the hydrogel.

The hydrogel of the present disclosure may also include an electrolyte or a mixture of electrolytes. The electrolyte may be a salt, such as lithium chloride, sodium chloride, potassium chloride, magnesium acetate, ammonium acetate, or any combination thereof. In embodiments, a suitable electrolyte may include potassium chloride. The hydrogel may possess the electrolyte in an amount from about 0.5 wt % to about 10 wt % of the hydrogel, in embodiments from about 1 wt % to about 8 wt % of the hydrogel. Electrolytes may be helpful where the hydrogel of the present disclosure is to be used as a conductive composition with an electrode.

The hydrogel of the present disclosure may also include a neutralizer. Bases such as hydroxides, amines, Lewis bases, and combinations thereof may be used as neutralizers. Non-limiting examples of neutralizers include ammonium hydroxide, sodium hydroxide, potassium hydroxide, lithium hydroxide, combinations thereof, and the like. If the acrylic acid and/or the second monomer, such as the 2-acrylamido-2-methylpropane sulfonic acid, are included as monomers in forming a copolymer for use in the hydrogel, it may be desirable to add neutralizer to neutralize some of the acid so that the pH of the mixture is from about 3 to about 6.5, in embodiments from about 3.5 to about 6, in other embodiments from about 4 to about 5.5.

Where utilized, a neutralizer may be present in an amount from about 1 wt % to about 8 wt % of the hydrogel, in embodiments from about 3 wt % to about 7 wt % of the hydrogel.

In some embodiments a thickener may be added to the hydrogel. Suitable thickeners include rheological modifiers which permit tailoring the viscosity of the hydrogel to permit its use as a drug delivery device and/or a conductive composition with a medical electrode. Non-limiting examples of such thickeners include silica, gums including xanthan gum, polymers including polyvinyl pyrrolidone (PVP), polyacrylamides, polyacrylic acid (including those sold under the name CARBOPOL®), salts thereof, combinations thereof, and the like. Where utilized, a thickener may be present in a hydrogel of the present disclosure in an amount from about 0.1 wt % to about 8 wt % of the hydrogel, in embodiments from about 0.5 wt % to about 5 wt % of the hydrogel.

In some embodiments, a suitable hydrogel of the present disclosure may include a copolymer. Non-limiting examples of suitable copolymers may include a first monomer, such as a mixture of acrylic acid and a salt thereof, and a second monomer, such as one or more monomers of the general formula CH₂═CHC(O)XR, in which X is O or NH, and R is an unsubstituted or substituted alkyl group of from about 1 to about 5 carbon atoms. The hydrogel may also include water; an electrolyte or mixture of electrolytes; a polymerization initiator; a neutralizer a such as sodium hydroxide; a penetration enhancer such as dimethylsulfoxide; optionally a humectant; optionally, a crosslinking agent; and optionally, a thickener.

An example of a suitable polymer which may be utilized in the hydrogel includes RG-63B, commercially available from Covidien. Other suitable hydrogels include those disclosed in U.S. Patent Application Publication Nos. 2009/0270709 and 2009/0270710, the entire disclosures of each of which are incorporated by reference herein. In embodiments, the above polymers and/or hydrogels may be modified in accordance with the present disclosure, rendering them suitable for use as drug delivery devices and/or use as conductive compositions with electrodes.

Other ingredients may be present in the hydrogel of the present disclosure. For example, humectants, penetration enhancers, and/or bioactive agents, may be added to a hydrogel of the present disclosure.

Water may also be present in the mixture utilized to form the hydrogel. The amount of water includes any water present in any of the ingredients and any water added with ingredients that are in water solution, such as the monomers, the crosslinking agent, the neutralizer, etc. In embodiments, humectants may be added to the water phase of a hydrogel of the present disclosure. Humectants which may be used include non-volatile, non-toxic, water soluble or water miscible liquids that are viscous at room temperature. Suitable humectants include, but are not limited to, polyhydric alcohols such as glycerol, sorbitol, ethylene glycol, propylene glycol, polyethylene glycols (PEG) of varying molecular weights including PEG 300, PEG 400 and PEG 600, polypropylene glycols, combinations thereof, and the like. The humectant may be utilized in combination with water or without water. Where utilized with water, the ratio of water to humectant may be from about 1:10 to about 10:1, in embodiments from about 2:8 to about 8:2.

As noted above, in use, a hydrogel of the present disclosure may contain the polymer or copolymer and any other additives described herein in an amount from about 4% by weight to about 97% by weight, in embodiments from about 20% by weight to about 60% by weight, with the balance being water and/or a humectant in an amount from about 3% to about 80% by weight of the hydrogel, in embodiments from about 6% by weight to about 10% by weight of the hydrogel.

In embodiments, a hydrogel of the present disclosure may include a penetration enhancer. The penetration enhancer may reduce the barrier effects of any tissue to which the hydrogel may be applied, including the skin and its various layers, including the stratum corneum, thereby enhancing the delivery rates and efficiencies of bioactive agents, including drugs, through tissue including the skin. The use of a penetration enhancer may thus allow for lower amounts and more even concentrations of drug(s) to be delivered over time, which may help mitigate any side effects attributable to the drug.

Any penetration enhancer within the purview of those skilled in the art may be added to a hydrogel of the present disclosure to aid in the delivery, in embodiments the transdermal delivery, of a bioactive agent. For example, in embodiments, sulfoxides such as dimethylsulfoxide (DMSO), decylmethyl sulfoxide and/or tetradecylmethyl sulfoxide, may be added to a hydrogel of the present disclosure. Other penetration enhancers which may be utilized include, but are not limited to, alcohols, including methanol, ethanol and 2-propanol; pyrrolidones, including 2-pyrrolidone, N-methyl-2-pyrrolidone and N-(2-hydroxyethyl)pyrrolidone; laurocapram; solvents such as acetone, dimethyl acetamide, dimethyl formamide, and tetrahydrofurfuryl alcohol; fatty alcohols, including lauryl alcohol; amides, including aromatic amides such as N,N-diethyl-m-toluamide; amino acids, including L-amino acids; azones; oils, including menthol or peppermint oil; fatty acids and their esters, including oleic acids, lauryl acids, isopropyl myristate, and glycerol monolaurate; macrocycles, including cyclopentadecanone; phospholipids, including lecithin; glycols, including ethoxy diglycol; combinations of any of the foregoing, and the like.

In embodiments, the bioactive agent may be soluble in the penetration enhancer, including DMSO. Materials, including bioactive agents, which are soluble in a penetration enhancer such as DMSO, may be readily carried through the skin without any damage to the skin.

In embodiments, a penetration enhancer such as DMSO may also function as a humectant. In such a case, the penetration enhancer may be used in addition to, or instead of, the humectants and/or water described above. For example, in some embodiments, a hydrogel that contains a humectant, such as glycerol, may include a penetration enhancer such as DMSO. The DMSO may also completely, or partially, replace the amount of humectant and/or water, such as glycerol, utilized in the hydrogel.

Where utilized, a penetration enhancer, such as DMSO, may be included in a hydrogel of the present disclosure in a suitable amount, in embodiments from about 2% to about 50% by weight of the hydrogel, in embodiments from about 5% to about 45% by weight of the hydrogel.

As noted above, in embodiments, a hydrogel of the present disclosure may be utilized to deliver a bioactive agent, such as a drug, to a patient. The drug delivery may be by any route of administration, including orally, buccally, intravenously, intramuscularly, parenterally, subcutaneously, sublingually, topically, combinations thereof, and the like. In embodiments, the hydrogel of the present disclosure may be applied topically and thus utilized for the transdermal administration of a bioactive agent, such as a drug. While the present disclosure sometimes refers to a hydrogel used in this manner as a “drug delivery device,” a “drug delivery device” in accordance with the present disclosure includes any delivery device that may be utilized to administer a bioactive agent, including a drug.

Suitable bioactive agents which may be administered by a hydrogel of the present disclosure include, for example, drugs, biocidal agents, antimicrobial agents, antibiotics, growth factors, anti-clotting agents, clotting agents, analgesics, including non-narcotic analgesics, anesthetics, including topical and/or local anesthetics, pain relievers, anti-inflammatory agents, wound repair agents, hormones, heart medications, nicotine, combinations thereof, and the like. Other bioactive agents which may be introduced with a hydrogel of the present disclosure include cosmeceuticals. As used herein, a “cosmeceutical” includes a topical cosmetic-pharmaceutic agent utilized to enhance the health of, and/or beauty of, the skin.

In embodiments, the bioactive agent may be an analgesic, such as methyl salicylate, salicylic acid, acetaminophen, oxycodone, hydrocodone, COX-2 inhibitors, non-steroidal anti-inflammatory drugs (NSAIDs), combinations thereof, and the like.

In embodiments, the bioactive agent may be an anesthetic such as benzocaine, bupivacaine, butesin picrate, chloroprocaine, ethyl chloride, fluori-methane, lidocaine HCl, mepivacaine, pramoxine HCl, combinations thereof, and the like.

In embodiments, the bioactive agent may be a cosmeceutical such as ace mannan, aloe powder, aloe vera gel, alpha-hydroxy acids, ammonium glycolate, α-bisabolol, ascorbic acid, beta-hydroxy acids, calamine, capsaicin, camphor, centella asiatica extract, dipotassium glycyrrhizinate, ginkgo biloba extract, ginseng extract, glucosamine, grape seed extract, green tea extract, horsetail extract, hydroquinone, kinetin, minoxidil, menthol, methyl sulfonyl methane, retinoic acid, vitamin A palmitate, vitamin E acetate, combinations thereof, and the like.

The bioactive agents may be administered to a subject in an effective amount. An effective amount is an amount which is capable of producing a desirable result in a treated animal or cell. As is well known in the medical and veterinary arts, a suitable dosage for any one animal depends on many factors, including the particular animal's size, body surface area, age, the particular composition to be administered, time and route of administration, general health, and other drugs or bioactive agents being administered concurrently. In embodiments, a bioactive agent may be present in an amount of from about 0.1% by weight of the hydrogel to about 20% by weight of the hydrogel, in embodiments from about 0.5% by weight of the hydrogel to about 10% by weight of the hydrogel.

Application and formation of a hydrogel of the present disclosure may be by any method, using any applicator or application system, within the purview of those skilled in the art. For example, in embodiments, sprayers and similar devices which allow for the components to be kept separate prior to application, but which permit mixing either upon expulsion from the sprayer, or upon contact with a substrate such as skin, may be utilized. Alternatively, the monomers and any additional components described above may be mixed and spread on the skin, and then allowed to polymerize, optionally by exposure to an initiator. The components may also be applied to a suitable substrate, including a bandage or film, or they may be coated as a layer on a release liner, for example a siliconized release substrate such as silicone coated polyethylene terephthalate film, or other substrate prior to polymerization. In other embodiments, the hydrogel may be formed, and then applied to tissue, including the skin, or applied to a substrate which, in turn, is then applied to tissue so that the hydrogel is adjacent thereto. Electrodes may be formed by conventional processes, such as application of a hydrogel to a roll or sheet. In other embodiments, hydrogels may be injected and cured, or dispensed and cured, optionally on some substrate, thereby forming an electrode.

Initiators which may be used in the polymerization process include those described above. Free radical polymerization may be initiated by, for example, heating the mixture when a thermal free radical polymerization initiator is present in the mixture, or exposing the mixture to actinic radiation when a photoinitiated free radical polymerization initiator is present in the mixture. Any convenient source or sources of actinic radiation providing wavelengths in the region of the spectrum that overlap the absorption bands of the photoinitiated free radical polymerization initiator can be used to activate polymerization. In some embodiments, ultraviolet light may be used. The radiation can also be natural or artificial, monochromatic or polychromatic, incoherent or coherent, and for high efficiency should correspond closely in wavelengths to the absorption bands of the polymerization initiator. Conventional light sources include fluorescent lamps, mercury vapor lamps, metal additive lamps, and arc lamps. Useful lasers are those whose emissions fall within or overlap the absorption bands of the photoinitiated free radical polymerization initiator. Although, if desired, the mixture may be degassed before polymerization and/or the polymerization may be carried out under an inert atmosphere, it is not necessary to degas the mixture before polymerization or to carry out the polymerization under an inert atmosphere. In embodiments, initiators including redox initiators, may be added to enhance the polymerization process. Suitable initiators include, but are not limited to, K₂S₂O₅, K₂S₂O₈, potassium persulfate, potassium sulfite, H₂O₂, benzophenone, combinations thereof, and the like. In other embodiments, an accelerator such as FeSO₄ may be added to increase the rate of polymerization.

### Medical Electrodes

As noted above, in embodiments, in addition to being suitable for drug delivery, hydrogels of the present disclosure may also be conductive, rendering them suitable for use as conductive compositions to be used with electrodes.

Medical electrodes transmit electrical signals or currents to or from a patient's skin and an external medical apparatus. Medical electrodes are within the purview of those skilled in the art. These electrodes may include a conductive composition including a hydrogel of the present disclosure on a substrate. The layer of conductive composition can be adhered to or contacted with the skin of the patient. The medical electrode may also include a conductive interface that is electrically connected to the layer of conductive composition and adapted to be electrically connected to an item of external medical equipment. For many applications, the conductive composition should be sufficiently adhesive to adhere to the patient's skin, i.e., be a conductive adhesive. The configuration of the electrode and the adhesive properties required will depend on the intended application, such as whether the electrode is a transmission electrode, i.e., an electrode that sends electric currents or signals to the patient's body, or a sensing or monitoring electrode, i.e., an electrode that sends electrical signals from the patient's body to external medical equipment.

Examples of suitable electrodes which may include hydrogels of the present disclosure as conductive compositions include those disclosed in U.S. Patent Application Publication Nos. 2010/0072060, 2009/0270709, 2009/0270710, and 2009/0227857, the entire disclosures of each of which are incorporated by reference herein for all purposes.

In some embodiments, the electric current developed by an electrode may further enhance the release and/or transdermal delivery of a bioactive agent of the present disclosure.

The following Examples are being submitted to illustrate embodiments of the present disclosure. These Examples are intended to be illustrative only and are not intended to limit the scope of the present disclosure. Also, parts and percentages are by weight unless otherwise indicated. As used herein, “room temperature” refers to a temperature of from about 20° C. to about 25° C.

### EXAMPLES 1-9

Hydrogel samples were prepared as follows. An RG-63B hydrogel, commercially available from Covidien LP (Mansfield, Mass.), was modified by replacing at least a portion of the glycerol component of the hydrogel with varying amounts of DMSO. The DMSO was substituted for the glycerol at substitution levels of 0, 5, 10, 25, 50, 75, 90, 95 and 100%. Each sample, having the varying substitution levels of DMSO, was designated Example 1-9, respectively. The corresponding DMSO concentrations in the resulting hydrogel formulation for each Example were 0, 2.18, 4.36, 10.9, 21.7, 32.69, 39.23, 41.41, and 43.59%, based on weight.

A summary of the amounts of DMSO substituted for glycerol in each Example, and the amounts of DMSO in the resulting hydrogels, are set forth in Table 1 below.

TABLE 1

| | Amount of DMSO | Amount of DMSO in |
|---|---|---|
| | substituted for glycerol in | resulting hydrogel (% by |
| Example | RG-63B hydrogel (%) | weight) |
| 1 | 0 | 0 |
| 2 | 5 | 2.18 |
| 3 | 10 | 4.36 |
| 4 | 25 | 10.9 |
| 5 | 50 | 21.7 |
| 6 | 75 | 32.69 |
| 7 | 90 | 39.23 |
| 8 | 95 | 41.41 |
| 9 | 100 | 43.59 |

All starting solutions polymerized well under UV light to yield self adhesive gels, which were also electrically conductive.

Adhesiveness of the gels was evaluated using a Texture Analyzer manufactured by Stable Micro Systems, Ltd. and obtained from Texture Technologies Corp. following the manufacturer's instructions. Briefly, the Texture Analyzer is connected to a computer, which possesses files tailored to the gel being tested. The Texture Analyzer also includes a probe, which is calibrated prior to each test. The calibration includes the probe height from the sample to be tested. For the hydrogels produced above, the calibration included placement of a sample of the hydrogel on a teflon sample position bar beneath the probe, which included multiple holes for placement of multiple samples of each hydrogel to be tested. A paper strip was also applied to the Teflon bar, the paper strip being about 1 inch×approximately 1 inch longer than the Teflon bar.

The sample to be tested was then prepared. A cutting tool was used to cut the hydrogel across the web into a sample size of 1 inch by the across web width. The release liner on the side opposite the side of the hydrogel to be tested was removed. For example, if the top side of the hydrogel was evaluated the bottom release liner was removed; if the bottom side of the hydrogel was evaluated, the top release liner was removed.

A strip of paper was applied over the exposed hydrogel so that there were no wrinkles in the sample, or any exposed hydrogel around the edges. The paper allowed the sample bar to slide easily in the base of the Texture Analyzer, and eliminated the need for cleaning the base.

The sample was turned over so that the paper was on the bottom, and the remaining release liner was removed.

The exposed hydrogel was applied to the bottom side of the Teflon sample bar, covering an appropriate number of the holes (test areas) in the bar. There were no wrinkles or puckers over any of the test areas.

The Teflon sample position bar was centered over the hole to be tested, so that the probe moved through the center of the hole in the Teflon sample position bar down to the test sample.

For the hydrogels of the above Examples, the probe height was about 10 mm. The probe was automatically lowered to the surface of the test sample, the test was run, and the probe elevated to a set position and stop.

The sample bar was advanced to the next access hole, centered under the probe and another sample was tested. This was repeated 10 times for each of the gels of Examples 1-9 above, i.e., 10 samples of each of the gels of Examples 1-9 were tested.

Tables 2-10 below contain the data obtained for each of the 10 samples of Examples 1-9, respectively, that were tested. The data includes the thickness of the gel, Force 1 (Initial Compression Force), Force 2 (Relaxed Compression Force), Force 3 (Primary Tack), Force 4 (Secondary Tack), Gradient-FT 1:2 (Grad.-FT 1:2) (Slope between F1 and F2), Area-FT 3:6 (Area under the entire curve), and Travel 3:6 (Leg length). The initial compression force was the force required to push the tip of the probe into the surface of the hydrogel to a depth of 0.3 mm. The relaxed compressive force was the force recorded after three seconds at the compressive depth of 0.3 mm. The primary adhesive force was the maximum force required to pull the probe away from the gel. The secondary adhesive force was the maximum force required to release residual polymer legs from the probe. The inflexion point was the force at which the gel failed and all gel was released from the probe. Travel 3:6 (leg length) was the maximum length the hydrogel legs stretched until failure.

TABLE 2

| | | | | | | | Grad.-FT | Area-FT | Travel |
|---|---|---|---|---|---|---|---|---|---|
| | | | | | | | 1:2 | 3:6 | 3:6 |
| | Product | Thickness | Force 1 | Force 2 | Force 3 | Force 4 | g/s | g · s | mm |
| Example 1 | Height | Mil | g | g | g | g | Grad.-FT | Area-FT | Travel |
| Sample | mm | E#/0.025 | Force 1 | Force 2 | Force 3 | Force 4 | 1:2 | 3:6 | 3:6 |
| 1 | 0.613 | 25 | −801.782 | −395.765 | 186.277 | 149.604 | 135.339 | 468.880 | 4.490 |
| 2 | 0.642 | 26 | −719.198 | −329.478 | 186.933 | 151.432 | 129.907 | 461.140 | 4.300 |
| 3 | 0.660 | 26 | −663.945 | −314.099 | 181.256 | 146.388 | 116.616 | 452.780 | 4.410 |
| 4 | 0.600 | 24 | −613.694 | −279.405 | 144.304 | 148.689 | 111.430 | 484.940 | 5.125 |
| 5 | 0.595 | 24 | −621.062 | −280.583 | 146.280 | 161.861 | 113.493 | 526.720 | 5.208 |
| 6 | 0.615 | 25 | −787.911 | −395.471 | 183.031 | 150.884 | 130.813 | 513.190 | 4.575 |
| 7 | 0.690 | 28 | −559.016 | −266.336 | 302.247 | 389.978 | 97.560 | 489.650 | 2.100 |
| 8 | 0.577 | 23 | −583.616 | −256.444 | 151.848 | 135.903 | 109.057 | 530.260 | 5.445 |
| 9 | 0.603 | 24 | −626.948 | −275.026 | 155.975 | 164.486 | 117.307 | 586.180 | 5.460 |
| 10 | 0.575 | 23 | −703.299 | −330.462 | 162.079 | 147.740 | 124.279 | 514.620 | 4.965 |
| Average | 0.617 | 25 | −668.047 | −312.307 | 180.023 | 174.697 | 118.580 | 502.837 | 4.608 |
| Std. | 0.037 | 1 | 82.997 | 50.796 | 46.122 | 76.057 | 11.577 | 40.007 | 0.979 |
| Dev. | | | | | | | | | |

TABLE 3

| | | | | | | | Grad.-FT | Area-FT | Travel |
|---|---|---|---|---|---|---|---|---|---|
| | | | | | | | 1:2 | 3:6 | 3:6 |
| | Product | Thickness | Force 1 | Force 2 | Force 3 | Force 4 | Grad.-FT | Area-FT | Travel |
| Example 2 | Height | Mil | g | g | g | g | g/s | g · s | mm |
| Sample | mm | E#/0.025 | Force 1 | Force 2 | Force 3 | Force 4 | 1:2 | 3:6 | 3:6 |
| 1 | 0.650 | 26 | −507.318 | −224.510 | 240.585 | 290.588 | 94.269 | 285.300 | 1.780 |
| 2 | 0.623 | 25 | −526.157 | −216.125 | 181.263 | 158.366 | 103.344 | 253.000 | 3.150 |
| 3 | 0.625 | 25 | −504.911 | −197.939 | 177.220 | 133.196 | 102.324 | 255.590 | 3.175 |
| 4 | 0.788 | 32 | −413.055 | −181.422 | 305.222 | 305.222 | 77.211 | 284.660 | 1.680 |
| 5 | 0.577 | 23 | −678.170 | −326.459 | 164.816 | 86.229 | 117.237 | 260.340 | 3.370 |
| 6 | 0.590 | 24 | −692.107 | −319.785 | 153.751 | 91.273 | 124.107 | 287.020 | 3.725 |
| 7 | 0.610 | 24 | −653.219 | −293.700 | 164.888 | 97.861 | 119.840 | 308.340 | 4.130 |
| 8 | 0.625 | 25 | −683.449 | −305.090 | 172.409 | 113.237 | 126.120 | 348.960 | 3.875 |
| 9 | 0.605 | 24 | −726.970 | −353.311 | 203.466 | 241.229 | 124.553 | 424.820 | 3.440 |
| 10 | 0.558 | 22 | −902.417 | −488.909 | 217.866 | 198.310 | 137.836 | 430.650 | 3.820 |
| Average | 0.625 | 25 | −628.777 | −290.725 | 198.148 | 171.551 | 112.684 | 313.868 | 3.215 |
| Std. | 0.063 | 3 | 142.095 | 91.916 | 46.256 | 82.871 | 18.100 | 66.305 | 0.843 |
| Dev. | | | | | | | | | |

TABLE 4

| | | | | | | | Grad.-FT | Area-FT | Travel |
|---|---|---|---|---|---|---|---|---|---|
| | | | | | | | 1:2 | 3:6 | 3:6 |
| | Product | Thickness | Force 1 | Force 2 | Force 3 | Force 4 | Grad.-FT | Area-FT | Travel |
| Example 3 | Height | Mil | g | g | g | g | g/s | g · s | mm |
| Sample | mm | E#/0.025 | Force 1 | Force 2 | Force 3 | Force 4 | 1:2 | 3:6 | 3:6 |
| 1 | 0.610 | 24 | −765.011 | −400.721 | 193.925 | 165.143 | 121.430 | 207.390 | 2.135 |
| 2 | 0.610 | 24 | −667.459 | −345.773 | 229.809 | 229.434 | 107.229 | 243.730 | 1.860 |
| 3 | 0.550 | 22 | −899.397 | −500.755 | 227.592 | 227.768 | 132.881 | 243.770 | 1.835 |
| 4 | 0.567 | 23 | −925.317 | −495.148 | 204.076 | 175.493 | 143.390 | 240.540 | 2.155 |
| 5 | 0.565 | 23 | −716.824 | −339.578 | 163.643 | 84.916 | 125.749 | 218.110 | 2.695 |
| 6 | 0.570 | 23 | −805.232 | −403.119 | 177.915 | 88.144 | 134.038 | 235.270 | 2.730 |
| 7 | 0.592 | 24 | −830.661 | −454.881 | 206.006 | 178.489 | 125.260 | 288.260 | 2.290 |
| 8 | 0.582 | 23 | −782.960 | −414.624 | 196.318 | 147.569 | 122.779 | 232.170 | 2.618 |
| 9 | 0.592 | 24 | −572.811 | −260.892 | 174.857 | 174.857 | 103.973 | 205.310 | 2.870 |
| 10 | 0.723 | 29 | −533.612 | −247.275 | 271.387 | 271.387 | 95.446 | 244.390 | 1.530 |
| Average | 0.596 | 24 | −749.928 | −386.277 | 204.553 | 174.320 | 121.217 | 235.892 | 2.272 |
| Std. | 0.048 | 2 | 129.124 | 88.081 | 31.769 | 59.212 | 14.865 | 23.659 | 0.448 |
| Dev. | | | | | | | | | |

TABLE 5

| | | | | | | | Grad.- | Area-FT | Travel |
|---|---|---|---|---|---|---|---|---|---|
| | | | | | | | FT 1:2 | 3:6 | 3:6 |
| | Product | Thickness | Force 1 | Force 2 | Force 3 | Force 4 | g/s | g · s | mm |
| Example 4 | Height | Mil | g | g | g | g | Grad.- | Area-FT | Travel |
| Sample | mm | E#/0.025 | Force 1 | Force 2 | Force 3 | Force 4 | FT 1:2 | 3:6 | 3:6 |
| 1 | 0.595 | 24 | −714.993 | −384.374 | 175.615 | 175.615 | 110.206 | 166.030 | 1.950 |
| 2 | 0.600 | 24 | −594.527 | −316.960 | 155.287 | 155.287 | 92.523 | 163.190 | 2.295 |
| 3 | 0.590 | 24 | −794.231 | −425.427 | 195.422 | 195.422 | 122.935 | 185.420 | 2.025 |
| 4 | 0.555 | 22 | −934.897 | −531.753 | 199.713 | 173.369 | 134.382 | 199.480 | 1.995 |
| 5 | 0.610 | 24 | −800.949 | −456.718 | 220.418 | 230.102 | 114.744 | 228.660 | 1.940 |
| 6 | 0.623 | 25 | −575.678 | −303.175 | 157.429 | 157.429 | 90.834 | 208.270 | 2.558 |
| 7 | 0.595 | 24 | −678.859 | −361.077 | 175.065 | 175.065 | 105.927 | 185.680 | 2.285 |
| 8 | 0.663 | 27 | −640.184 | −314.008 | 178.180 | 178.180 | 108.725 | 209.540 | 2.355 |
| 9 | 0.595 | 24 | −734.212 | −393.581 | 198.087 | 160.769 | 113.544 | 188.260 | 2.205 |
| 10 | 0.645 | 26 | −731.327 | −371.491 | 189.917 | 189.917 | 119.945 | 206.750 | 2.245 |
| Average | 0.607 | 24 | −719.986 | −385.856 | 184.513 | 179.116 | 111.376 | 194.128 | 2.185 |
| Std. | 0.030 | 1 | 107.100 | 71.103 | 20.136 | 22.160 | 13.199 | 20.403 | 0.203 |
| Dev. | | | | | | | | | |

TABLE 6

| | | | | | | | Grad.-FT | Area-FT | Travel |
|---|---|---|---|---|---|---|---|---|---|
| | | | | | | | 1:2 | 3:6 | 3:6 |
| | Product | Thickness | Force 1 | Force 2 | Force 3 | Force 4 | g/s | g · s | mm |
| Example 5 | Height | Mil | g | g | g | g | Grad.-FT | Area-FT | Travel |
| Sample | mm | E#/0.025 | Force 1 | Force 2 | Force 3 | Force 4 | 1:2 | 3:6 | 3:6 |
| 1 | 0.630 | 25 | −483.506 | −213.125 | 146.605 | 160.369 | 90.127 | 218.050 | 2.390 |
| 2 | 0.853 | 34 | −221.028 | −92.051 | 196.221 | 196.221 | 42.992 | 175.820 | 1.650 |
| 3 | 0.785 | 31 | −312.579 | −128.018 | 212.578 | 212.578 | 61.520 | 233.240 | 1.845 |
| 4 | 0.620 | 25 | −552.476 | −255.786 | 136.361 | 137.651 | 98.897 | 246.030 | 3.110 |
| 5 | 0.585 | 23 | −546.435 | −224.867 | 139.483 | 80.534 | 107.189 | 233.750 | 3.630 |
| 6 | 0.665 | 27 | −502.213 | −210.834 | 139.986 | 85.504 | 97.126 | 265.590 | 3.248 |
| 7 | 0.695 | 28 | −449.882 | −202.164 | 167.509 | 162.538 | 82.573 | 271.170 | 2.683 |
| 8 | 0.860 | 34 | −233.610 | −99.111 | 169.113 | 169.113 | 44.833 | 212.790 | 2.395 |
| 9 | 0.610 | 24 | −647.976 | −298.927 | 156.613 | 101.451 | 116.350 | 276.890 | 3.325 |
| 10 | 0.527 | 21 | −584.980 | −226.587 | 129.802 | 81.522 | 119.465 | 262.280 | 4.205 |
| Average | 0.683 | 27 | −453.468 | −195.147 | 159.427 | 138.748 | 86.107 | 239.561 | 2.848 |
| Std. Dev. | 0.114 | 5 | 148.728 | 67.738 | 27.276 | 48.990 | 27.794 | 31.523 | 0.802 |

TABLE 7

| | | | | | | | Grad.-FT | Area- | Travel |
|---|---|---|---|---|---|---|---|---|---|
| | | | | | | | 1:2 | FT 3:6 | 3:6 |
| | Product | Thickness | Force 1 | Force 2 | Force 3 | Force 4 | g/s | g · s | mm |
| Example 6 | Height | Mil | g | G | g | g | Grad.-FT | Area- | Travel |
| Sample | mm | E#/0.025 | Force 1 | Force 2 | Force 3 | Force 4 | 1:2 | FT 3:6 | 3:6 |
| 1 | 0.613 | 25 | −484.593 | −246.794 | 118.611 | 118.611 | 79.267 | 180.070 | 2.700 |
| 2 | 0.618 | 25 | −484.605 | −247.005 | 114.414 | 114.414 | 79.200 | 190.590 | 3.035 |
| 3 | 0.663 | 27 | −606.020 | −334.467 | 159.598 | 106.359 | 90.518 | 249.550 | 2.790 |
| 4 | 0.635 | 25 | −566.798 | −285.935 | 136.718 | 80.853 | 93.621 | 208.720 | 2.960 |
| 5 | 0.678 | 27 | −629.738 | −344.151 | 164.499 | 161.744 | 95.196 | 267.420 | 2.665 |
| 6 | 0.660 | 26 | −440.408 | −226.127 | 113.696 | 79.696 | 71.427 | 211.840 | 3.280 |
| 7 | 0.875 | 35 | −282.675 | −135.092 | 144.480 | 159.018 | 49.194 | 273.470 | 2.905 |
| 8 | 0.777 | 31 | −302.652 | −149.770 | 115.886 | 88.628 | 50.961 | 245.040 | 3.565 |
| 9 | 0.750 | 30 | −339.747 | −156.957 | 108.418 | 89.929 | 60.930 | 256.480 | 3.765 |
| 10 | 0.822 | 33 | −383.973 | −192.308 | 128.955 | 120.971 | 63.888 | 305.810 | 3.460 |
| Average | 0.709 | 28 | −452.121 | −231.861 | 130.528 | 112.022 | 73.420 | 238.897 | 3.113 |
| Std. Dev. | 0.092 | 4 | 124.256 | 74.405 | 20.063 | 29.616 | 16.945 | 39.978 | 0.384 |

TABLE 8

| | | | | | | | Grad.-FT | Area- | Travel |
|---|---|---|---|---|---|---|---|---|---|
| | | | | | | | 1:2 | FT 3:6 | 3:6 |
| | Product | Thickness | Force 1 | Force 2 | Force 3 | Force 4 | g/s | g · s | mm |
| Example 7 | Height | Mil | g | g | g | g | Grad.-FT | Area- | Travel |
| Sample | mm | E#/0.025 | Force 1 | Force 2 | Force 3 | Force 4 | 1:2 | FT 3:6 | 3:6 |
| 1 | 0.563 | 23 | −472.640 | −233.234 | 127.645 | 105.323 | 79.802 | 245.670 | 3.215 |
| 2 | 0.658 | 26 | −545.584 | −302.485 | 115.525 | 107.143 | 81.033 | 286.140 | 3.575 |
| 3 | 0.735 | 29 | −452.954 | −231.685 | 113.648 | 113.226 | 73.756 | 313.010 | 3.650 |
| 4 | 0.697 | 28 | −259.240 | −116.370 | 71.578 | 70.617 | 47.623 | 239.450 | 4.635 |
| 5 | 0.715 | 29 | −231.078 | −102.218 | 73.455 | 79.587 | 42.953 | 255.590 | 4.435 |
| 6 | 0.858 | 34 | −326.024 | −164.818 | 158.580 | 141.955 | 53.735 | 357.100 | 3.220 |
| 7 | 0.752 | 30 | −232.572 | −96.033 | 90.954 | 90.684 | 45.513 | 250.380 | 3.975 |
| 8 | 0.835 | 33 | −38.319 | −9.196 | 60.996 | 60.304 | 9.708 | 204.010 | 4.843 |
| 9 | 0.945 | 38 | −277.540 | −130.613 | 116.659 | 129.544 | 48.975 | 337.150 | 3.645 |
| 10 | 0.720 | 29 | −325.215 | −144.981 | 116.478 | 110.370 | 60.078 | 301.930 | 3.785 |
| Average | 0.748 | 30 | −316.117 | −153.163 | 104.552 | 100.875 | 54.318 | 279.044 | 3.898 |
| Std. Dev. | 0.108 | 4 | 146.123 | 84.014 | 29.964 | 25.668 | 21.199 | 48.166 | 0.567 |

TABLE 9

| | | | | | | | Grad.- | Area-FT | Travel |
|---|---|---|---|---|---|---|---|---|---|
| | | | | | | | FT 1:2 | 3:6 | 3:6 |
| | Product | Thickness | Force 1 | Force 2 | Force 3 | Force 4 | g/s | g · s | mm |
| Example 8 | Height | Mil | g | g | g | g | Grad.- | Area-FT | Travel |
| Sample | mm | E#/0.025 | Force 1 | Force 2 | Force 3 | Force 4 | FT 1:2 | 3:6 | 3:6 |
| 1 | 0.527 | 21 | −371.093 | −164.205 | 93.494 | 82.438 | 68.963 | 268.370 | 4.585 |
| 2 | 0.540 | 22 | −584.302 | −300.694 | 137.971 | 100.163 | 94.536 | 282.220 | 3.500 |
| 3 | 0.572 | 23 | −434.347 | −205.453 | 102.739 | 92.411 | 76.298 | 281.680 | 4.238 |
| 4 | 0.558 | 22 | −497.341 | −228.833 | 115.515 | 86.956 | 89.503 | 256.310 | 3.765 |
| 5 | 0.567 | 23 | −503.878 | −212.662 | 120.760 | 91.932 | 97.072 | 259.450 | 3.405 |
| 6 | 0.538 | 22 | −547.530 | −257.439 | 114.566 | 89.090 | 96.697 | 248.010 | 3.505 |
| 7 | 0.533 | 21 | −602.578 | −305.546 | 126.648 | 95.428 | 99.011 | 246.270 | 3.213 |
| 8 | 0.560 | 22 | −522.482 | −262.954 | 111.981 | 89.355 | 86.509 | 230.120 | 3.590 |
| 9 | 0.522 | 21 | −458.172 | −219.173 | 97.191 | 76.745 | 79.666 | 213.550 | 3.725 |
| 10 | 0.490 | 20 | −374.441 | −160.847 | 89.630 | 76.230 | 71.198 | 220.080 | 3.833 |
| Average | 0.541 | 22 | −489.617 | −231.781 | 111.050 | 88.075 | 85.945 | 250.607 | 3.736 |
| Std. | 0.025 | 1 | 80.124 | 50.161 | 15.361 | 7.733 | 11.231 | 23.900 | 0.408 |
| Dev. | | | | | | | | | |

TABLE 10

| | | | | | | | Grad.- | Area-FT | Travel |
|---|---|---|---|---|---|---|---|---|---|
| | | | | | | | FT 1:2 | 3:6 | 3:6 |
| | Product | Thickness | Force 1 | Force 2 | Force 3 | Force 4 | g/s | g · s | mm |
| Example 9 | Height | Mil | g | g | g | g | Grad.- | Area-FT | Travel |
| Sample | mm | E#/0.025 | Force 1 | Force 2 | Force 3 | Force 4 | FT 1:2 | 3:6 | 3:6 |
| 1 | 0.680 | 27 | −213.640 | −96.884 | 77.365 | 85.513 | 38.919 | 291.800 | 4.790 |
| 2 | 0.700 | 28 | −353.075 | −172.753 | 96.786 | 106.107 | 60.107 | 320.720 | 4.375 |
| 3 | 0.730 | 29 | −355.196 | −179.294 | 97.468 | 102.368 | 58.634 | 313.030 | 4.200 |
| 4 | 0.635 | 25 | −338.914 | −155.449 | 84.699 | 79.611 | 61.155 | 257.280 | 4.310 |
| 5 | 0.673 | 27 | −346.143 | −154.261 | 89.979 | 92.171 | 63.961 | 276.850 | 4.195 |
| 6 | 0.635 | 25 | −334.089 | −148.983 | 81.927 | 87.062 | 61.702 | 269.840 | 4.365 |
| 7 | 0.598 | 24 | −336.891 | −148.866 | 80.250 | 81.809 | 62.675 | 258.860 | 4.570 |
| 8 | 0.603 | 24 | −343.703 | −151.458 | 82.981 | 83.743 | 64.082 | 264.380 | 4.223 |
| 9 | 0.595 | 24 | −312.687 | −136.703 | 76.622 | 81.264 | 58.661 | 257.730 | 4.570 |
| 10 | 0.730 | 29 | −352.566 | −154.189 | 107.308 | 88.667 | 66.126 | 284.540 | 3.935 |
| Average | 0.658 | 26 | −328.690 | −149.884 | 87.538 | 88.832 | 59.602 | 279.503 | 4.353 |
| Std. | 0.052 | 2 | 42.283 | 22.220 | 10.094 | 8.972 | 7.658 | 22.907 | 0.242 |
| Dev. | | | | | | | | | |

The gels were placed on tab electrodes for testing. The electrical conductivity of the gels was evaluated using an Electrode Tester from AngioLaz, Inc. (Bellows Falls, Vt., USA), having a dedicated computer attached thereto. The Electrode Tester was an electronic instrument designed to test for compliance to the Electrical Performance per American National Standards Institute/Association for the Advancement of Medical Instrumentation (ANSI/AAMI) EC 12:2000, “Disposable ECG Electrodes.”

Twelve electrode pairs were connected to each set of lead wires. The Electrode tester tested the DC Offset, AC Impedance, SDR Max Volts, and SDR Recovery Slope for the electrode. Electrode pairs that passed the test were identified, as were those that did not pass the test. 12 electrodes were tested for each of the gels of Examples 1-9 above.

Tables 11-19 below contain the data obtained for the 12 sample electrodes prepared with the gels of Examples 1-9 above, respectively. (DCO (Direct Current Offset), slope (Recovery Slope), and ACZ (Alternating Current Impedance)) are provided. Table 20 includes a summary of results for each gel of Examples 1-9.

TABLE 11
Electrode Electrical Test Results
Example 1

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | Ohm | mv | mv | ohm | Noise |
| 1 | 0.621 | 340 | 8.83 | 0.111 | 270 | 0.040 |
| 2 | 1.300 | 318 | 8.18 | 0.109 | 264 | 0.018 |
| 3 | 0.845 | 316 | 8.76 | 0.112 | 267 | 0.011 |
| 4 | 0.833 | 369 | 9.13 | 0.107 | 288 | 0.020 |
| 5 | 1.830 | 329 | 8.41 | 0.110 | 263 | 0.076 |
| 6 | 0.787 | 337 | 9.18 | 0.110 | 283 | 0.023 |
| 7 | 0.266 | 337 | 9.58 | 0.114 | 289 | 0.008 |
| 8 | 1.291 | 309 | 8.28 | 0.111 | 262 | 0.024 |
| 9 | 0.793 | 373 | 9.08 | 0.110 | 310 | 0.023 |
| 10 | 5.400 | 317 | 7.57 | 0.115 | 268 | 0.013 |
| 11 | 0.380 | 345 | 10.40 | 0.110 | 249 | 0.029 |
| 12 | 0.786 | 315 | 8.56 | 0.108 | 273 | 0.018 |
| Total | 15.132 | 4005 | 105.96 | 1.327 | 3286 | 0.303 |
| Average | 1.261 | 334 | 8.83 | 0.111 | 274 | 0.025 |
| Varience | 1.370 | 21 | 0.73 | 0.002 | 16 | 0.018 |
| AAMI* | 100 mv | 2 Kohm | 100 mv | 1 mv/sec | 2 KOhm | |
| Lim. | max | max | max | max | max | |

*Association for the Advancement of Medical Instrumentation Limits

TABLE 12
Electrode Electrical Test Results
Example 2

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | om | mv | mv | ohm | Noise |
| 1 | 2.320 | 282 | 10.30 | 0.103 | 242 | 0.014 |
| 2 | 0.795 | 287 | 8.88 | 0.114 | 252 | 0.036 |
| 3 | 0.736 | 304 | 8.76 | 0.116 | 274 | 0.019 |
| 4 | 0.685 | 305 | 9.14 | 0.108 | 255 | 0.065 |
| 5 | 0.405 | 314 | 9.28 | 0.105 | 241 | 0.026 |
| 6 | 0.882 | 322 | 8.85 | 0.111 | 276 | 0.015 |
| 7 | 0.046 | 324 | 9.80 | 0.105 | 258 | 0.024 |
| 8 | 1.733 | 314 | 7.98 | 0.113 | 276 | 0.017 |
| 9 | 1.052 | 319 | 8.42 | 0.116 | 285 | 0.035 |
| 10 | 0.940 | 331 | 8.82 | 0.108 | 263 | 0.069 |
| 11 | 0.704 | 294 | 9.03 | 0.108 | 236 | 0.015 |
| 12 | 1.518 | 324 | 8.12 | 0.106 | 279 | 0.015 |
| Total | 11.816 | 3720 | 107.38 | 1.313 | 3137 | 0.350 |
| Average | 0.985 | 310 | 8.95 | 0.109 | 261 | 0.029 |
| Varience | 0.613 | 16 | 0.65 | 0.004 | 17 | 0.019 |
| AAMI | 100 mv | 2 Kohm | 100 mv | 1 mv/sec | 2 KOhm | |
| Lim. | max | max | max | max | max | |

TABLE 13
Electrode Electrical Test Results
Example 3

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | ohm | mv | mv | ohm | Noise |
| 1 | 0.246 | 306 | 8.93 | 0.115 | 248 | 0.039 |
| 2 | 0.562 | 318 | 8.86 | 0.110 | 276 | 0.018 |
| 3 | 0.170 | 360 | 9.65 | 0.109 | 280 | 0.011 |
| 4 | 0.627 | 356 | 8.83 | 0.116 | 274 | 0.021 |
| 5 | 0.363 | 311 | 9.31 | 0.112 | 260 | 0.013 |
| 6 | 0.677 | 325 | 8.53 | 0.109 | 271 | 0.020 |
| 7 | 0.502 | 303 | 8.82 | 0.115 | 255 | 0.022 |
| 8 | 1.136 | 324 | 8.25 | 0.108 | 271 | 0.023 |
| 9 | 0.585 | 338 | 9.40 | 0.106 | 263 | 0.022 |
| 10 | 1.945 | 332 | 7.81 | 0.114 | 268 | 0.112 |
| 11 | 1.017 | 306 | 8.58 | 0.120 | 242 | 0.028 |
| 12 | 1.258 | 366 | 8.06 | 0.109 | 292 | 0.015 |
| Total | 9.088 | 3945 | 105.03 | 1.343 | 3200 | 0.344 |
| Average | 0.757 | 329 | 8.75 | 0.112 | 267 | 0.029 |
| Varience | 0.504 | 22 | 0.55 | 0.004 | 14 | 0.027 |
| AAMI | 100 mv | 2 Kohm | 100 mv | 1 mv/sec | 2 KOhm | |
| Lim. | max | max | max | max | max | |

TABLE 14
Electrode Electrical Test Results
Example 4

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | ohm | mv | mv | ohm | Noise |
| 1 | 0.283 | 305 | 9.01 | 0.111 | 256 | 0.036 |
| 2 | 0.879 | 308 | 8.42 | 0.109 | 263 | 0.014 |
| 3 | 0.337 | 327 | 9.13 | 0.111 | 279 | 0.021 |
| 4 | 0.566 | 297 | 8.73 | 0.111 | 252 | 0.017 |
| 5 | 0.513 | 301 | 8.92 | 0.110 | 258 | 0.012 |
| 6 | 0.954 | 306 | 8.33 | 0.113 | 274 | 0.018 |
| 7 | 0.691 | 291 | 8.52 | 0.106 | 248 | 0.015 |
| 8 | 1.849 | 298 | 7.60 | 0.113 | 259 | 0.021 |
| 9 | 0.900 | 404 | 8.28 | 0.108 | 257 | 0.020 |
| 10 | 1.914 | 331 | 7.35 | 0.109 | 283 | 0.011 |
| 11 | 0.617 | 279 | 8.76 | 0.112 | 225 | 0.038 |
| 12 | 1.348 | 279 | 7.91 | 0.108 | 246 | 0.013 |
| Total | 10.851 | 3726 | 100.96 | 1.321 | 3100 | 0.236 |
| Average | 0.904 | 311 | 8.41 | 0.110 | 258 | 0.020 |
| Varience | 0.541 | 33 | 0.56 | 0.002 | 16 | 0.009 |
| AAMI | 100 mv | 2 Kohm | 100 mv | 1 mv/sec | 2 KOhm | |
| Lim. | max | max | max | max | max | |

TABLE 15
Electrode Electrical Test Results
Example 5

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | Ohm | mv | mv | ohm | Noise |
| 1 | 0.111 | 283 | 8.41 | 0.114 | 263 | 0.034 |
| 2 | 0.719 | 268 | 7.30 | 0.107 | 250 | 0.011 |
| 3 | 0.309 | 304 | 7.67 | 0.105 | 279 | 0.030 |
| 4 | 0.633 | 267 | 7.44 | 0.105 | 246 | 0.014 |
| 5 | 0.270 | 264 | 8.57 | 0.110 | 238 | 0.010 |
| 6 | 1.154 | 294 | 6.77 | 0.101 | 291 | 0.017 |
| 7 | 0.973 | 292 | 8.83 | 0.110 | 275 | 0.010 |
| 8 | 1.471 | 291 | 6.60 | 0.107 | 277 | 0.018 |
| 9 | 0.335 | 286 | 7.85 | 0.111 | 273 | 0.016 |
| 10 | 0.343 | 263 | 7.58 | 0.108 | 243 | 0.011 |
| 11 | 0.509 | 240 | 8.41 | 0.109 | 218 | 0.023 |
| 12 | 1.131 | 275 | 6.71 | 0.107 | 264 | 0.010 |
| Total | 7.958 | 3327 | 92.14 | 1.294 | 3117 | 0.204 |
| Average | 0.663 | 277 | 7.68 | 0.108 | 260 | 0.017 |
| Varience | 0.430 | 18 | 0.76 | 0.003 | 21 | 0.008 |
| AAMI | 100 mv | 2 Kohm | 100 mv | 1 mv/sec | 2 KOhm | |
| Lim. | max | max | max | max | max | |

TABLE 16
Electrode Electrical Test Results
Example 6

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | ohm | mv | mv | ohm | Noise |
| 1 | 0.814 | 253 | 7.69 | 0.109 | 240 | 0.032 |
| 2 | 2.365 | 283 | 5.17 | 0.104 | 267 | 0.009 |
| 3 | 0.506 | 289 | 7.20 | 0.110 | 278 | 0.015 |
| 4 | 0.169 | 280 | 7.07 | 0.104 | 268 | 0.013 |
| 5 | 0.040 | 250 | 7.05 | 0.105 | 235 | 0.008 |
| 6 | 0.479 | 288 | 6.87 | 0.100 | 284 | 0.015 |
| 7 | 0.293 | 285 | 7.11 | 0.104 | 270 | 0.011 |
| 8 | 0.693 | 264 | 6.12 | 0.109 | 256 | 0.016 |
| 9 | 0.447 | 262 | 6.32 | 0.106 | 252 | 0.014 |
| 10 | 0.989 | 280 | 5.91 | 0.104 | 265 | 0.014 |
| 11 | 1.201 | 255 | 5.57 | 0.099 | 239 | 0.020 |
| 12 | 0.456 | 257 | 6.24 | 0.106 | 247 | 0.009 |
| Total | 8.452 | 3246 | 78.32 | 1.260 | 3101 | 0.176 |
| Average | 0.704 | 271 | 6.53 | 0.105 | 258 | 0.015 |
| Varience | 0.619 | 15 | 0.75 | 0.003 | 16 | 0.006 |
| AAMI | 100 mv | 2 Kohm | 100 mv | 1 mv/sec | 2 KOhm | |
| Lim. | max | max | max | max | max | |

TABLE 17
Electrode Electrical Test Results
Example 7

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | ohm | mv | mv | ohm | Noise |
| 1 | 0.615 | 264 | 6.47 | 0.090 | 257 | 0.032 |
| 2 | 0.870 | 281 | 5.33 | 0.088 | 268 | 0.009 |
| 3 | 0.936 | 276 | 6.10 | 0.095 | 268 | 0.007 |
| 4 | 0.440 | 273 | 6.22 | 0.093 | 261 | 0.016 |
| 5 | 0.842 | 271 | 5.34 | 0.084 | 260 | 0.009 |
| 6 | 0.633 | 281 | 6.11 | 0.098 | 283 | 0.014 |
| 7 | 0.424 | 288 | 6.11 | 0.092 | 279 | 0.010 |
| 8 | 2.382 | 267 | 4.16 | 0.089 | 261 | 0.018 |
| 9 | 1.449 | 285 | 4.87 | 0.091 | 280 | 0.015 |
| 10 | 2.001 | 303 | 5.38 | 0.103 | 288 | 0.009 |
| 11 | 0.384 | 241 | 5.56 | 0.092 | 229 | 0.020 |
| 12 | 1.226 | 281 | 5.46 | 0.093 | 275 | 0.009 |
| Total | 12.202 | 3311 | 67.11 | 1.108 | 3209 | 0.168 |
| Average | 1.017 | 276 | 5.59 | 0.092 | 267 | 0.014 |
| Varience | 0.641 | 15 | 0.66 | 0.005 | 16 | 0.007 |
| AAMI | 100 mv | 2 Kohm | 100 mv | 1 mv/sec | 2 KOhm | |
| Lim. | max | max | max | max | max | |

TABLE 18
Electrode Electrical Test Results
Example 8

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | ohm | mv | mv | ohm | Noise |
| 1 | 0.205 | 273 | 6.71 | 0.092 | 256 | 0.033 |
| 2 | 0.071 | 280 | 6.58 | 0.097 | 268 | 0.011 |
| 3 | 0.835 | 280 | 5.77 | 0.084 | 269 | 0.008 |
| 4 | 0.049 | 277 | 6.41 | 0.086 | 257 | 0.066 |
| 5 | 1.748 | 284 | 5.31 | 0.101 | 270 | 0.010 |
| 6 | 0.401 | 274 | 5.74 | 0.096 | 280 | 0.017 |
| 7 | 0.231 | 290 | 6.31 | 0.088 | 280 | 0.011 |
| 8 | 1.744 | 265 | 5.09 | 0.092 | 259 | 0.015 |
| 9 | 1.447 | 272 | 5.47 | 0.099 | 263 | 0.016 |
| 10 | 1.240 | 279 | 5.56 | 0.104 | 258 | 0.010 |
| 11 | 0.209 | 237 | 6.34 | 0.102 | 227 | 0.021 |
| 12 | 0.805 | 271 | 6.27 | 0.104 | 265 | 0.046 |
| Total | 8.985 | 3282 | 71.56 | 1.145 | 3152 | 0.264 |
| Average | 0.749 | 274 | 5.96 | 0.095 | 263 | 0.022 |
| Varience | 0.650 | 13 | 0.54 | 0.007 | 14 | 0.018 |
| AAMI | 100 mv | 2 Kohm | 100 mv | 1 mv/sec | 2 KOhm | |
| Lim. | max | max | max | max | max | |

TABLE 19
Electrode Electrical Test Results
Example 9

| | DC | | Defibrillation Overload | | | |
|---|---|---|---|---|---|---|
| | Offset | Impedance | Recovery | | | |
| | Voltage | ACZ | DCO | Slope | ACZ | |
| Sample # | mv | Ohm | mv | mv | ohm | Noise |
| 1 | 1.670 | 254 | 8.47 | 0.104 | 252 | 0.034 |
| 2 | 0.195 | 261 | 6.36 | 0.099 | 254 | 0.097 |
| 3 | 0.796 | 269 | 7.83 | 0.109 | 264 | 0.007 |
| 4 | 0.236 | 278 | 7.67 | 0.103 | 276 | 0.017 |
| 5 | 1.490 | 281 | 8.03 | 0.092 | 273 | 0.008 |
| 6 | 0.285 | 292 | 6.56 | 0.106 | 288 | 0.018 |
| 7 | 2.000 | 252 | 7.77 | 0.093 | 239 | 0.010 |
| 8 | 0.868 | 271 | 4.98 | 0.091 | 266 | 0.016 |
| 9 | 0.708 | 210 | 5.42 | 0.097 | 203 | 0.023 |
| 10 | 1.042 | 284 | 6.97 | 0.122 | 273 | 0.012 |
| 11 | 0.700 | 243 | 7.91 | 0.116 | 230 | 0.023 |
| 12 | 0.338 | 225 | 5.97 | 0.103 | 225 | 0.008 |
| Total | 10.328 | 3120 | 83.94 | 1.235 | 3043 | 0.273 |
| Average | 0.861 | 260 | 7.00 | 0.103 | 254 | 0.023 |
| Varience | 0.593 | 25 | 1.13 | 0.010 | 25 | 0.025 |
| AAMI | 100 mv | 2 Kohm | 100 mv | 1 mv/sec | 2 KOhm | |
| Lim. | max | max | max | max | max | |

TABLE 20
Summary Results

| | | | | | Defibrillation |
|---|---|---|---|---|---|
| | | | | | Overload |
| | | | | Impedance | Recovery |
| | | Force 3 | Force 4 | ACZ | Slope |
| Example | % DMSO | g | g | ohm | Mv |
| 1 | 0 | 180.023 | 174.697 | 334 | 0.111 |
| 2 | 2.18 | 198.148 | 174.551 | 310 | .0109 |
| 3 | 4.36 | 204.553 | 174.320 | 329 | 0.112 |
| 4 | 10.9 | 184.513 | 179.006 | 311 | 0.110 |
| 5 | 21.7 | 159.427 | 138.748 | 277 | 0.108 |
| 6 | 32.69 | 130.528 | 112.022 | 271 | .0105 |
| 7 | 39.23 | 104.522 | 100.875 | 276 | 0.092 |
| 8 | 41.41 | 111.050 | 88.075 | 274 | 0.095 |
| 9 | 43.59 | 87.538 | 88.832 | 260 | 0.103 |

As can be seen from the average results of Examples 1-9 listed in Table 20, the presence of DMSO had little impact on the adhesive qualities of the hydrogel. A DMSO content of 10.9% resulted in an average primary adhesive force of about 184 grams and an average secondary adhesive force of about 179 grams, while the hydrogel without any DMSO had an average primary adhesive force of about 180 grams and an average secondary adhesive force of about 175 grams. Increasing the content of DMSO to 39.23% only slightly reduced the adhesive strength. A hydrogel having a DMSO content of 29.23% had an average primary adhesive force of 104.522 grams and an average secondary adhesive strength of 100.875 grams. The hydrogel having a DMSO content of 43.49% showed an additional decrease in the average primary and secondary adhesive strength; however, these adhesive strengths remained within acceptable parameters for use. Therefore, the addition of DMSO did not substantially affect the effectiveness of adhesion of the hydrogel.

As can also be seen from the average results of Examples 1-9 listed in Table 20, the presence of DMSO increased conductivity. As the DMSO content was increased from zero to 43.59%, the impedance dropped from 334 ohms to 260 ohms. Similarly, as the DMSO content was increased from zero to 43.59%, the slope of the defibrillation overload recovery dropped from 0.111 ohms to 0.103 ohms. Therefore, the addition of DMSO improved the conductivity of the hydrogel.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, or material.

What is claimed is:

1. A medical electrode comprising:
a substrate;
a conductive composition on at least a portion of a surface of the substrate, the conductive composition comprising at least one hydrogel comprising:
a polymeric component comprising a copolymer comprising a first monomer comprising a mixture of acrylic acid and a salt thereof, present in an amount of from about 8 weight % to about 85 weight % of the copolymer, and a second monomer of the formula CH₂═CHC(O)XR, in which X is O or NH and R is an unsubstituted or substituted alkyl group of from about 1 to about 5 carbon atoms present in an amount of from about 15 weight % to about 92 weight % of the copolymer;
at least one penetration enhancer selected from the group consisting of sulfoxides, alcohols, pyrrolidones, laurocapram, solvents, fatty alcohols, amides, amino acids, azones, oils, fatty acids and their esters, macrocycles, phospholipids, glycols, and combinations thereof; and
at least one bioactive agent.

2. The medical electrode of claim 1, wherein the penetration enhancer is selected from the group consisting of dimethylsulfoxide, decylmethyl sulfoxide, tetradecylmethyl sulfoxide, methanol, ethanol, 2-propanol, 2-pyrrolidone, N-methyl-2-pyrrolidone, N-(2-hydroxyethyl)pyrrolidone, acetone, dimethyl acetamide, dimethyl formamide, tetrahydrofurfuryl alcohol, lauryl alcohol, N,N-diethyl-m-toluamide, L-amino acids, menthol, peppermint oil, oleic acids, lauryl acids, isopropyl myristate, glycerol monolaurate, cyclopentadecanone, lecithin, ethoxy diglycol, and combinations thereof, present in an amount of from about 2% to about 50% by weight of the hydrogel.

3. The medical electrode of claim 1, wherein the bioactive agent is selected from the group consisting of cosmeceuticals, drugs, biocidal agents, antimicrobial agents, antibiotics, growth factors, anti-clotting agents, clotting agents, analgesics, anesthetics, pain relievers, anti-inflammatory agents, wound repair agents, hormones, heart medications, nicotine, and combinations thereof.

4. The medical electrode of claim 1, wherein the bioactive agent comprises an analgesic selected from the group consisting of methyl salicylate, salicylic acid, acetaminophen, oxycodone, hydrocodone, COX-2 inhibitors, non-steroidal anti-inflammatory drugs, and combinations thereof.

5. The medical electrode of claim 1, wherein the bioactive agent comprises an anesthetic selected from the group consisting of benzocaine, bupivacaine, butesin picrate, chloroprocaine, ethyl chloride, fluori-methane, lidocaine HCl, mepivacaine, pramoxine HCl, and combinations thereof.

6. The medical electrode of claim 1, wherein the bioactive agent comprises a cosmeceutical selected from the group consisting of ace mannan, aloe powder, aloe vera gel, alpha-hydroxy acids, ammonium glycolate, α-bisabolol, ascorbic acid, beta-hydroxy acids, calamine, capsaicin, camphor, centella asiatica extract, dipotassium glycyrrhizinate, ginkgo biloba extract, ginseng extract, glucosamine, grape seed extract, green tea extract, horsetail extract, hydroquinone, kinetin, minoxidil, menthol, methyl sulfonyl methane, retinoic acid, vitamin A palmitate, vitamin E acetate, and combinations thereof.

7. The medical electrode of claim 1, wherein the bioactive agent is present in an amount of from about 0.1% by weight of the hydrogel to about 20% by weight of the hydrogel.

8. The medical electrode of claim 1, wherein the hydrogel further comprises a humectant selected from the group consisting of glycerol, sorbitol, ethylene glycol, propylene glycol, polyethylene glycol, polypropylene glycol, and combinations thereof.

9. The medical electrode of claim 8, wherein the humectant, optionally in combination with water, is present in an amount of from about 3% to about 80% by weight of the hydrogel.

10. The medical electrode of claim 1, wherein the hydrogel further comprises an electrolyte present in an amount of from about 0.5% by weight to about 10% by weight of the hydrogel, and optionally a neutralizer selected from the group consisting of ammonium hydroxide, sodium hydroxide, potassium hydroxide, lithium hydroxide, and combinations thereof, optionally a cross linking agent selected from the group consisting of N—N′-methylene bis-acrylamide, diethylene glycol diacrylate, diethylene glycol dimethacrylate, trimethylolpropane triacrylate, trimethylolpropane trimethacrylate, ethoxylated trimethylolpropane triacrylate, ethoxylated trimethylolpropane trimethacrylate, pentaerythritol triacrylate, pentaerythritol trimethacrylate, pentaerythritol tetraacrylate, pentaerythritol tetramethacrylate, polyethylene glycol diacrylate, polyethylene glycol dimethacrylate, and combinations thereof, and optionally a polymerization initiator selected from the group consisting of 2,2-azobisisobutyronitrile, 1-hydroxycyclohexylphenyl ketone, 2-hydroxy-2-methyl-1-phenylpropan-1-one, 2-hydroxy-1-[4-(2-hydroxyethoxy)phenyl]-2-methyl-1-propan-1-one, 2,2-dimethoxy-2-phenylacetophenone, benzophenone, and combinations thereof.
